(19) United States
(12) Patent Application Publication
(10) Pub. No.: US 2006/0194972 A1
(43) Pub. Date: Aug. 31, 2006

(54) Process for the production of imidazopyridin-8-ones

(75) Inventors: Daniel Mink, EUPEN (BE); Paulus Lambertus Alsters, Maastricht (NL)

Correspondence Address: NATH & ASSOCIATES PLLC, 112 South West Street, Alexandria, VA, 22314, US

(73) Assignee: ALTANA PHARMA AG, KONSTANZ (DE)

(21) Appl. No.: 10/550,691

(22) PCT Filed: Apr. 1, 2004
(86) PCT No.: PCT/EP04/50414

§ 371 Date: Sep. 26, 2005

(30) Foreign Application Priority Data
Apr. 3, 2003 (EP) 03007663.2

(51) Int. Cl. A61K 31/4745; C07D 471/14

(52) U.S. Cl. 546/82

(57) ABSTRACT

The invention relates to a process for the production of 7-(trialkyl-silanytoxy)-2,3-dimethyl-8-phenyl-8,9dihydro-7H-1,3a,9-triazacyclopenta[a]naphthalen-6-one and related compounds by using NBS as oxidizing agent.

### FIELD OF APPLICATION OF THE INVENTION

The invention relates to a novel process, which is used in the pharmaceutical industry in the synthesis of intermediates for the production of medicaments.

### PRIOR ART

The international patent applications WO98/42707, WO01/72756, WO01/172757 and WO02/34749 disclose tricyclic imidazopyridine derivatives having a very specific substitution pattern, which are suited for the treatment of gastric and intestinal disorders. In said patent applications, reaction schemes are given in which the synthesis of the final products, starting from imidazopyridin-8-ones, is illustrated. These imidazopyridin-8-ones are described in more detail in international patent application WO01/72748. In several publications, such as Karmakar et al., Journal of the American Chemical Society 77, 55-69 (1955), Zechmeister et al., Journal of the American Chemical Society 75, 4493-4495 (1953) and Snyder et al., Journal of the American Chemical Society 71, 1395-1396 (1949) the use of N-bromosuccinimide in dehydrogenation processes is described.

### DESCRIPTION OF THE INVENTION

The invention relates to a process, which is used for the preparation of important intermediates for the production of the compounds mentioned in the prior art, and further compounds having a similar basic structure.

The invention relates in a first aspect to a process for the production of compounds of formula 1,

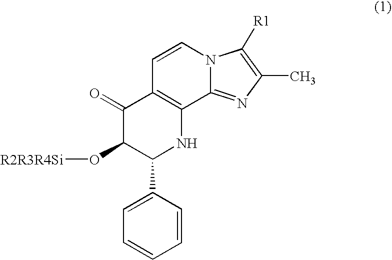

in which

R1 is hydrogen, methyl or hydroxymethyl,

R2 is 1-7C-alkyl,

R3 is 1-7C-alkyl and

R4 is 1-7C-alkyl,

and their salts.

1-7C-Alkyl represents straight-chain or branched alkyl radicals having 1 to 7 carbon atoms. Examples which may be mentioned are the heptyl radical, isoheptyl radical (5-methylhexyl radical), hexyl radical, isohexyl radical (4-methylpentyl radical), neohexyl radical (3,3-dimethylbutyl radical), pentyl radical, isopentyl radical (3-methylbutyl radical), neopentyl radical (2,2-dimethylpropyl radical), butyl radical, isobutyl radical, sec-butyl radical, tert-butyl radical, propyl radical, isopropyl radical, ethyl radical and the methyl radical.

Suitable salts of compounds of the formula 1 are especially all acid addition salts. Particular mention may be made of the salts of the inorganic and organic acids customarily used. Those which are suitable are water-soluble and water-insoluble acid addition salts with acids such as hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, maleic acid, fumaric acid, succinic acid, oxalic acid, tartaric acid, embonic acid, stearic acid, toluene-sulfonic acid, methanesulfonic acid or 3-hydroxy-2-naphthoic acid, where the acids are employed in salt preparation—depending on whether it is a mono- or polybasic acid and depending on which salt is desired—in an equimolar quantitative ratio or one differing there from.

It is known to the person skilled in the art that the compounds according to the invention and their salts, if they are isolated, for example, in crystalline form, can contain various amounts of solvents. The invention therefore also comprises all solvates and in particular all hydrates of the compounds of the formula 1, and also all solvates and in particular all hydrates of the salts of the compounds of the formula 1.

The process is characterized in that compounds of formula 2,

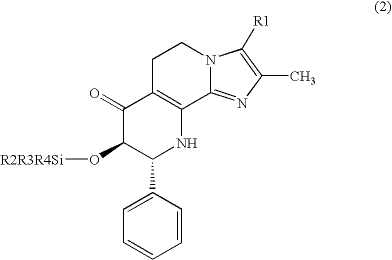

in which R1, R2, R3 and R4 have the meanings given above, are dehydrogenated (oxidized) with NBS (N-bromosuccinimide).

The dehydrogenation (oxidation) with NBS is carried out in an inert solvent, for example in a chlorinated hydrocarbon, such as carbon tetrachloride or dichloromethane, or in a ketone, e. g. acetone or butanone, or in an ether, e. g. tetrahydrofuran or dioxan, or in DMSO or in acetontrile.

The reaction of NBS with a compound of formula 2 is conveniently effected at a temperature of −70° C. to +50° C., preferably at a temperature of 0° C. to +30° C., and with the subsequent aid of a base, preferably with an organic base, such as an amine, e. g. diisopropylamine, methyl-diisopropylamine or, in particular, triethylamine. Advantageously, NBS is added to a solution of the compound of formula 2 in a first step, using an amount of 1,0 equivalents of NBS, with immediate subsequent start of the addition of the base.

Preferred compounds of formula 1, which are prepared by the process according to the invention, are those, in which

R1 is methyl,

R2 is 1-7C-alkyl,

R3 is 1-4C-alkyl and

R4 is 1-4C-alkyl,

and their salts.

Particularly preferred compounds of formula 1, which are prepared by the process according to the invention, are those, in which

R1 is methyl,

R2 is tert-butyl,

R3 is methyl and

R4 is methyl,

and their salts.

The starting compounds of formula 2 can be prepared, according to the following reaction scheme.

Scheme

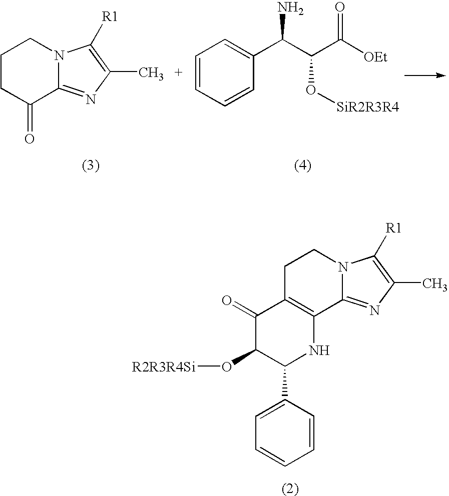

The starting compound of formula (3) is known from WO01/72748. The silyl ether of formula (4) can be prepared according to methods known to the expert, for example by reacting phenylisoserine ethyl ester with tert-butyl-dimethylsilyl chloride under basic conditions. The reaction of (3) and (4) is preferably carried out in the presence of a suitable catalyst, for example p-toluenesulfonic acid, and under simultaneous removal of water. The initial formation of an intermediate imine is followed by a ring closure, which is performed by using a strong base, for example potassium tert-butylate, lithium tert-butylate, sodium bis(trimethylsilyl)amide or preferably lithium diisopropylamide.

The compounds of formula 1 are valuable intermediates for the synthesis of compounds as described in international patent applications WO98/42707 and WO01/72756. The 8-hydroxy-7-oxo-7,8,9,10-tetrahydroimidazo[1,2-h][1,7]naphthyridine, which is given for example in scheme 8 of international patent application WO98/42707 as intermediate, is obtained from compounds 1 by hydrolysis, for example with hydrochloric acid.

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s) and RT for room temperature.

### EXAMPLES

### 1. t-Butyl-dimethyl-silylether of phenyl isoserine ethyl ester

1323 g (4.06 mole) of (R,R)-phenylisoserine ethyl ester are dissolved in 6.6. L of dichloromethane. To this solution, 397.4 g of imidazole and 724 g of t-butyldimethylsilyl chloride are added. The mixture is stirred for 16 h at RT. The reaction mixture is washed subsequently with 6 L and 4 L of water. The resulting clear dichloromethane layer is dried over sodium sulphate, filtered and concentrated under reduced pressure. The obtained 1509 g of the title compound are used as such in Example 2 without further purification.

### 2. 7-(t-Butyl-dimethyl-silanyloxy)-2,3-dimethyl-8-phenyl-5,7,8,9-tetrahydro-4H-1,3a,9-triaza-cyclopenta[a]naphthalen-6-one

To 1509 g of t-butyl-dimethyl-silylether of phenyl isoserine ethyl ester (obtained in Example 1), dissolved in 10.5 L of toluene, 14 g of p-toluenesulphonic acid monohydrate and 736 g of 2,3-dimethyl-6,7-dihydro-5H-imidazo[1,2-a]pyridin-8-one are added. The mixture is stirred and boiled under reflux until 80 mL of water are collected in the Dean-Stark trap used. The mixture is cooled to −15° C. and 6 L of THF are added. To this solution, 6 L of 2 M lithium-diisopropylamide (solution in THF/n-heptane) are added dropwise within 1 h. The mixture is stirred for 30 min. without external cooling (the temperature rises to −5° C.) and then quenched with 7 L of aqueous ammonium chloride solution. The two layers are separated. The organic layer is dried over sodium sulphate and filtered. After removal of the solvents in vacuo, 1811 g of crude 7-(tertbutyl-dimethyl-silanyloxy)-2,3-dimethyl-8-phenyl-5,7,8,9-tetrahydro-4H-1,3a,9-triaza-cyclopenta[a]naphthalen-6-one are isolated. This material is dissolved in 3.9 L of boiling methanol and cooled to −5° C. while stirring. The formed precipitate is collected and rinsed with 1.75 L of cold methanol. After drying, 558 g of the title compound are obtained. The mother liquor is concentrated to 1.5 L and stirred at −5° C. for several hours. The precipitate is collected and rinsed with 0.25 L of methanol. Another portion of 96.5 g of the title compound are isolated. Total yield is 654.5 g (38.5%).

### 3. 7-(t-Butyl-dimethyl-silanyloxy)-2,3-dimethyl-8-phenyl-8,9-dihydro-7H-1,3a,9-triaza-cyclopenta[a]naphthalen-6-one

25 g (59.1 mmole) of 7-(tertbutyl-dimethyl-silanyloxy)-2,3-dimethyl-8-phenyl-5,7,8,9-tetrahydro-4H-1,3a,9-triaza-cyclopenta[a]naphthalen-6-one are suspended in 150 ml of acetonitrile. The mixture is stirred and cooled in a thermostated reactor at 15° C. A solution of 10.52 g (1 equivalent) of N-bromosuccinimide in 100 ml of acetonitrile is added in the course of 1 h while keeping the temperature, at 15° C. When addition of N-bromosuccinimide is completed, 22.5 ml of triethylamine are added with further stirring at 15° C. within the course of 45 min. Stirring is continued for additional 2 h at 15° C. After cooling the obtained suspension to 10° C., 138 ml of water are added slowly during 30 min. The suspension is cooled to 5° C., stirred for further 30 min and then filtered. The yellow filter cake is washed twice with 125 ml of methanol/water 85:15 v/v and then dried. The tie compound is obtained as a yellow solid.

### 4. 7-Hydroxy-2,3-dimethyl-8-phenyl-8,9-dihydro-7H-1,3a,9-triaza-cyclopenta[a]naphthalen-6-one

386.5 g (0.916 mole) of 7-(t-butyl-dimethyl-silanyloxy)-2,3-dimethyl-8-phenyl-8,9-dihydro-7H-1,3a,9-triaza-cyclopenta[a]naphthalen-6-one are suspended in 1.4 L of methanol and cooled on an ice/water bath to 10° C. Then 0.734 L of 30% aqueous hydrochloride solution are added. The suspension becomes clear and after a few seconds a new precipitate is formed. The resulting suspension is stirred for two hours. After addition of 1.1 L of 25% aqueous ammonia the basic suspension (pH=9.6) is stirred for 1 hour. The formed solid is collected and rinsed with 1.1 L water and dried. To remove remaining silyl starting material, the solid is rinsed with 1 L of diethyl ether and dried again. 273.5 g of the title compound are obtained.

1. Process for the production of compounds of formula 1, 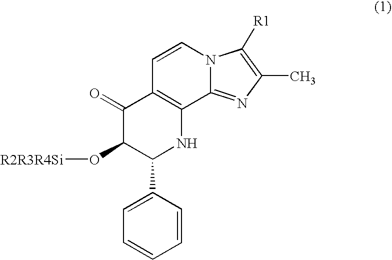
in which
R1 is hydrogen, methyl or hydroxymethyl,
R2 is 1-7C-alkyl,
R3 is 1-7C-alkyl and
R4 is 1-7C-alkyl,
and their salts,
which comprises dehydrogenating (oxidizing) compounds of formula 2, 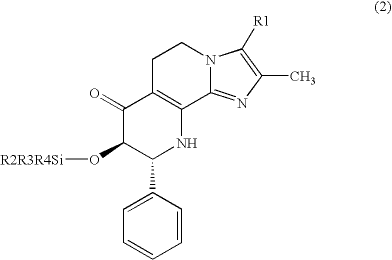
in which R1, R2, R3 and R4 have the meanings given above, by using NBS (N-bromosuccinimide).

2. Process as claimed in claim 1, for the production of compounds of formula 1, in which
R1 is methyl,
R2 is bromine,
R2 is 1-7C-alkyl,
R3 is 1-4C-alkyl and
R4 is 1-4C-alkyl.

3. Process as claimed in claim 1, for the production of compounds of formula 1, in which
R1 is methyl,
R2 is bromine,
R2 is tertbutyl,
R3 is methyl and
R4 is methyl.

4. Process as claimed in claim 1, characterized in that the amount of NBS used is approximately 1 equivalent, calculated on the basis of the amount of the compound of formula 2 used.

5. Process as claimed in claim 1, characterized in that subsequent to the reaction with NBS an organic base is used for the removal of HBr.

6. Process as claimed in claim 1 characterized in that subsequent to the reaction with NBS an organic amine is used for the removal of HBr.

7. Process as claimed in claim 1, characterized in that subsequent to the reaction with NBS triethylamine is used for the removal of HBr.

8. Process as claimed in claim 1, characterized in that the reaction is effected at a temperature of −70° C. to +50° C.

9. Process as claimed in claim 1, characterized in that the reaction is effected at a temperature of 0° C. to +30° C.

10. Process as claimed in claim 1, characterized in that the reaction is effected in an inert organic solvent.
